(19) United States
(12) Patent Application Publication
(10) Pub. No.: US 2021/0283093 A1
(43) Pub. Date: Sep. 16, 2021

(54) COMPOSITIONS COMPRISING BENZOYL PEROXIDE FOR THE TREATMENT OF GASTROINTESTINAL DISORDER

(71) Applicant: Sol-Gel Technologies Ltd., Ness Ziona (IL)

(72) Inventors: Ori NOV, Tarum (IL); Karine NEIMANN, Ness Ziona (IL); Asher CACHLON, Ashkelon (IL)

(73) Assignee: Sol-Gel Technologies Ltd., Ness Ziona (IL)

(21) Appl. No.: 17/202,394

(22) Filed: Mar. 16, 2021

Related U.S. Application Data
(60) Provisional application No. 62/989,932, filed on Mar. 16, 2020.

(51) Int. Cl. A61K 31/327; A61K 9/00; A61K 45/06; A61K 9/06; A61P 33/00

(52) U.S. Cl. CPC A61K 31/327; A61K 9/0014; A61P 33/00; A61K 9/06; A61K 45/06

(57) ABSTRACT

The present invention relates to the treatment, prevention or alleviation of a gastrointestinal disorder caused by one or more intestinal helminths, said treatment, prevention or alleviation comprises administering a topical composition comprising a peroxide to a subject in need thereof.

### CROSS REFERENCE TO RELATED APPLICATIONS

This application claims the benefit of U.S. Ser. No. 62/989,932, filed on Mar. 16, 2020, which is incorporated in its entirety herein by reference.

### FIELD OF THE INVENTION

The present invention relates to the treatment, prevention or alleviation of a gastrointestinal disorder caused by one or more intestinal helminths, said treatment, prevention or alleviation comprises administering a topical composition comprising a peroxide to a subject in need thereof.

### BACKGROUND OF THE INVENTION

Parasitic worms (helminths) including pinworm are common in children. Pinworm infection (called enterobiasis or oxyuriasis) is the most common type of intestinal worm. Infection occurs most often in school-age children when the Pinworms microscopic eggs are easily spread from child to child. Pinworms are thin and white, measuring about 6 to 13 millimeters in length.

Symptoms are caused by the female pinworm laying her eggs in the folds of skin surrounding the anus while the infected subject is asleep. Symptoms of pinworm infection are usually mild and some infected people have no symptoms. Among the symptoms of pinworms infection are itching of the anal or vaginal area, intermittent abdominal pain and nausea, insomnia, irritability and restlessness.

Pinworm eggs become infective within a few hours after being deposited on the skin around the anus and can survive for 2 to 3 weeks on clothing, bedding, or other objects. (Cochrane Database Syst Rev. 2001 April; 2001(2)). That is why, all household contacts and caretakers of the infected person should be treated at the same time.

Pinworm can be treated with either prescription or over-the-counter medications. The prescribed medications comprise Mebendazole and Albendazole (Albenza). Treatments can involve two doses of medication with the second dose being given 2 weeks after the first dose. However, there is an increased awareness of the potential problem of anthelmintic resistance (AR) in the treatment and control of human helminths.

Benzoyl peroxide has an antibacterial, irritant, keratolytic, comedolytic, and anti-inflammatory activity, and is used for the treatment of acne. Benzoyl peroxide decomposes to release ROS (reactive oxygen species) which are lethal to bacteria and other organisms.

### SUMMARY OF THE INVENTION

The present invention provides a method of treating, preventing and/or ameliorating a gastrointestinal disorder caused by one or more intestinal helminths, comprising administering topically to a subject in need thereof a therapeutically effective amount of a topical composition comprising a peroxide and a carrier suitable for topical administration. In another embodiment, the gastrointestinal disorder is enterobiasis.

The present invention also provides a regimen of administration for treating, preventing or ameliorating a gastrointestinal disorder caused by one or more intestinal helminths, wherein the regimen comprises at least two doses of administration of a topical composition comprising a peroxide and a carrier suitable for topical administration, wherein the consecutive dose is administered within 7-14 days following the previous administration, until remission. In another embodiment, the gastrointestinal disorder is enterobiasis.

The present invention provides a regimen of administration for treating, preventing or ameliorating a gastrointestinal disorder caused by one or more intestinal helminths, wherein the regimen comprises two doses of administration of a topical composition comprising a peroxide and a carrier suitable for topical administration, wherein the consecutive dose is administered within 7-14 days following the previous administration, until remission. In another embodiment, the gastrointestinal disorder is enterobiasis.

### DETAILED DESCRIPTION OF THE INVENTION

### The Composition

In some embodiments, the present invention provides a topical composition for use in treating, preventing and/or ameliorating of a gastrointestinal disorder caused by one or more intestinal helminths, wherein the composition comprises a peroxide and a carrier suitable for topical administration.

In one embodiment, the present invention provides a topical peroxide composition for use in treating, preventing and/or ameliorating of a gastrointestinal disorder caused by intestinal helminths, wherein the peroxide is benzoyl peroxide. In another embodiment, the gastrointestinal disorder is a pinworm infection (enterobiasis).

In another embodiment, the peroxide is in an amount of about 1% w/w to about 10.0% w/w of the composition. In another embodiment, the peroxide is in an amount of between 1% w/w to about 5% w/w of the composition. In another embodiment, the peroxide is in an amount of between 3% w/w to about 7% w/w of the composition. In another embodiment, the peroxide is in an amount of between 4% w/w to about 10% w/w of the composition. In another embodiment, the peroxide is in an amount of 3%, 5% or 7% w/w of the composition. In another embodiment, the peroxide is benzoyl peroxide. In another embodiment, the benzoyl peroxide is in an amount of about 1% w/w to about 10.0% w/w of the composition. In another embodiment, the benzoyl peroxide is in an amount of between 1% w/w to about 5% w/w of the composition of the composition. In another embodiment, the benzoyl peroxide is in an amount of between 3% w/w to about 7% w/w of the composition. In another embodiment, the benzoyl peroxide is in an amount of between 4% w/w to about 10% w/w of the composition. In another embodiment, the benzoyl peroxide is in an amount of 3%, 5% or 7% w/w of the composition. In another embodiment, the benzoyl peroxide is in an amount of 3% w/w of the composition. In another embodiment, the benzoyl peroxide is in an amount of 5% w/w of the composition. In another embodiment, the benzoyl peroxide is in an amount of 7% w/w of the composition.

In some embodiments, the topical composition for use in treating, preventing and/or ameliorating of a gastrointestinal disorder caused by one or more intestinal helminths, comprises a peroxide and a carrier suitable for topical administration. In another embodiment, the gastrointestinal disorder is enterobiasis.

In other embodiments, the carrier of the composition is water, mineral oil, olive oil, petrolatum, silicon oil, paraffin, medium chain triglyceride (MCT), beeswax, castor oil, organic oil such as coconut oil or mixtures thereof. In another embodiment, the carrier is water.

In another embodiment, the composition described herein is in a dosage form selected from a cream, a lotion, a gel, an ointment, an emulsion, a solution, a suspension, an elixir, a tincture, a paste, a foam, an aerosol, a spray, patch, transdermal patch and an applicator syringe. In another embodiment, the composition is a cream.

In some embodiments, the composition of the present invention further comprises excipients. In another embodiment, the excipients are selected from the group consisting of a gelling agent, an emulsifier, emollient, a stiffing agent, a humectant, a preservative and any combination thereof. Each represents a separate embodiment of this invention.

In another embodiment, a non-limiting example of an emulsifier is selected from sodium lauryl sulphate, disodium dioctyl sulfosuccinate, glyceryl stearate, PEG-100 stearate, stearyl alcohol, cetyl alcohol, laureth-23, steareth alcohol, cetyl/PEG/PPG 10 dimethicone, stearic acid and any combination thereof. Each represents a separate embodiment of this invention. In another embodiment, the emulsifier is between 5-25% of the composition. In another embodiment, the emulsifier is between 10-25% of the composition. In another embodiment, the emulsifier is between 15-25% of the composition.

In another embodiment, a non-limiting example of an emollient is selected from mineral oil, isopropyl myristate, urea, glycerol, propylene glycol, lactic acid and any combination thereof. Each represents a separate embodiment of this invention. In another embodiment, the emollient is between 10-30% w/w of the composition. In another embodiment, the emollient is between 10-20% w/w of the composition. In another embodiment, the emollient is between 15-25% w/w of the composition.

In another embodiment, a non-limiting example of a preservative is selected from propyl paraben, methyl paraben, benzalkonium chloride, benzoic acid, benzyl alcohol, imidazolidinyl urea, sodium benzoate, sorbic acids, thimerosal and any combination thereof. Each represents a separate embodiment of this invention. In another embodiment, the preservative is between 0.01-0.3% w/w of the composition. In another embodiment, the preservative is between 0.01-0.2% w/w of the composition. In another embodiment, the preservative is between 0.02-0.2% w/w of the composition. In another embodiment, the preservative is between 0.05-0.2% w/w of the composition.

In another embodiment, a non-limiting example of a humectant is selected from the group consisting of sorbitol, propylene glycol, polypropylene glycol, glycerin, hexylene and butylene glycol, urea, collagen, and any combination thereof. Each represents a separate embodiment of this invention. In another embodiment, the preservative is between 1-10% w/w of the composition. In another embodiment, the preservative is between 3-7% w/w of the composition.

In another embodiment, a non-limiting example of a gelling agent is selected from the group consisting of acacia, alginic acid, bentonite, Carbopols (known as carbomers), carboxymethyl cellulose, ethylcellulose, gelatin, hydroxyethyl cellulose, hydroxypropyl cellulose, magnesium aluminum silicate (Veegum), methylcellulose, poloxamers (Pluronics), polyvinyl alcohol, sodium alginate, tragacanth, and xanthan gum and any combination thereof. Each represents a separate embodiment of this invention.

In another embodiment, a non-limiting example of a stiffening agent is selected from the group consisting of cetyl alcohol, cetostearyl alcohol, parrafin and wax or any combination thereof. Each represents separate embodiment of this invention. In another embodiment, the stiffening agent is between 1-10% w/w of the composition. In another embodiment, the stiffening agent is between 5-10% w/w of the composition.

Pharmaceutical carriers or vehicles suitable for administration of the compounds provided herein include any such carriers known to those skilled in the art to be suitable for the particular mode of administration. The active agents are included in the carrier in an amount sufficient to exert a therapeutically useful effect i.e., amelioration of the symptoms of the gastrointestinal disorder is, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

In another embodiment, the composition is a cream comprising benzoyl peroxide (1-10% w/w), mineral oil (10-20% w/w), stearic acid (5-15% w/w), cetyl alcohol (5-10% w/w), sorbitol (3-10% w/w), PEG (3-10% w/w), isopropyl myristate (3-10% w/w), propyl paraben/methyl paraben (0.1-0.5 w/w) and water as a carrier.

The present invention also provides a composition comprising benzoyl peroxide (BPO) for use in treating, preventing and/or ameliorating pinworm infection (enterobiasis) in a cream or suppositories.

In another embodiment, the composition is a cream as described in Example 1.

In some embodiments, the peroxide of the composition is either encapsulated or non-encapsulated. In another embodiment, the peroxide is benzoyl peroxide, and the benzoyl peroxide is encapsulated. In another embodiment, the peroxide is benzoyl peroxide, and the benzoyl peroxide is non-encapsulated. In yet another embodiment, the benzoyl peroxide is encapsulated by silica.

The treatment with encapsulated benzoyl peroxide is advantageous as being less irritant in sensitive and mucosal areas like the anus. The above compositions are useful for the treatment, prevention or alleviation of a gastrointestinal disorder. The above compositions are useful for the treatment, prevention or alleviation of intestinal helminths. The above compositions are useful for the treatment, prevention or alleviation of enterobiasis.

In some other embodiment, the composition is formulated for extended release and wherein the extended-release effect is obtained by encapsulation, microencapsulation, microspheres or coating.

The encapsulation of benzoyl peroxide and compositions thereof for treatment of gastrointestinal disorder caused by one or more intestinal helminths are disclosed in a Sol-Gel Technologies Ltd. patent and a patent application, U.S. Pat. No. 9,687,465 and published U.S. Patent Application No. 2018147165, whose contents are enclosed herein in their entirety.

In another embodiment, the composition of the present invention further comprises at least one additional active agent. In some embodiments, the additional active agent is selected from Ivermectin, Mebendazole, Albendazole, pyrantel pamoate, Praziquantel, niclosamide, metrifonate, oxamniquine, bithionol, vermicides, vermifuges, anti-mite agent and any combination thereof. Each represents a separate embodiment of this invention.

### Methods of Treatment

A further aspect of the invention is a method of treatment, prevention or alleviation of a gastrointestinal disorder caused by one or more intestinal helminths, comprising administering topically to a subject in need thereof a therapeutically effective amount of a topical composition comprising a peroxide and a carrier suitable for topical administration. In another embodiment, the gastrointestinal disorder is a pinworm infection (enterobiasis).

In some embodiments, this invention provides a method of treating, preventing and/or ameliorating of a gastrointestinal disorder caused by one or more intestinal helminths, comprising administering a topical composition comprising a peroxide and a carrier suitable for topical administration. In another embodiment, the gastrointestinal disorder is a pinworm infection (enterobiasis).

In some embodiments, this invention provides a method of treatment, prevention or alleviation of a gastrointestinal disorder caused by one or more intestinal helminths, comprising administering topically to a subject in need thereof a topical composition as described herein. In another embodiment, the gastrointestinal disorder is enterobiasis.

In one embodiment, this invention provides a method of treatment, prevention or alleviation of a gastrointestinal disorder caused by one or more intestinal helminths, comprising administering to a subject in need thereof a topical composition comprising a peroxide in an amount of about 1% w/w to about 10.0% w/w of the composition. In another embodiment, the peroxide is in an amount of between 1% w/w to about 5% w/w of the composition. In another embodiment, the peroxide is in an amount of between 3% w/w to about 7% w/w of the composition. In another embodiment, the peroxide is in an amount of between 4% w/w to about 10% w/w of the composition. In another embodiment, the peroxide is in an amount of 3%, 5% or 7% w/w of the composition. In another embodiment, the peroxide is benzoyl peroxide. In another embodiment, the benzoyl peroxide is in an amount of between 1% w/w to about 10% w/w of the composition. In another embodiment, the benzoyl peroxide is in an amount of between 1% w/w to about 5% w/w of the composition. In another embodiment, the peroxide is in an amount of between 3% w/w to about 7% w/w of the composition. In another embodiment, the peroxide is in an amount of between 4% w/w to about 10% w/w of the composition. In another embodiment, the benzoyl peroxide is in an amount of 3% w/w of the composition. In another embodiment, the benzoyl peroxide is in an amount of 5% w/w of the composition. In another embodiment, the benzoyl peroxide is in an amount of 7% w/w of the composition. In another embodiment, the gastrointestinal disorder is enterobiasis.

In some embodiments, this invention provides a method of treating, preventing and/or ameliorating gastrointestinal disorder caused by one or more intestinal helminths, comprising administering topical composition comprising a peroxide and a carrier suitable for topical administration in combination with at least one additional active agent.

In another embodiment the additional active agent is selected from Ivermectin, Mebendazole, Albendazole, pyrantel pamoate, Praziquantel, niclosamide, metrifonate, oxamniquine, bithionol, vermicides, vermifuges, anti-mite agents, and any combination thereof. In another embodiment, the additional active agent is administered systematically.

In some other embodiments, there is provided a method of treating, preventing, alleviating of a gastrointestinal disorder caused by intestinal helminths in a patient in need thereof with benzoyl peroxide, said method comprising topically administering to an intestinal helminths affected skin surface of said patient a therapeutically effective amount of a composition comprising from about 1% w/w to about 10% w/w or from about 3% w/w to about 7% w/w benzoyl peroxide and a pharmaceutically acceptable carrier or excipient. In another embodiment, the gastrointestinal disorder is enterobiasis.

In some other embodiments, there is provided a method of treating, preventing, alleviating intestinal helminths in a patient in need thereof with benzoyl peroxide, said method comprising topically administering to an intestinal helminths affected skin surface of said patient a therapeutically effective amount of a composition comprising from about 1% w/w to about 10% w/w or from about 3% w/w to about 7% w/w benzoyl peroxide and a pharmaceutically acceptable carrier or excipient.

In some other embodiments, there is provided a method of treating, preventing, alleviating of a gastrointestinal disorder caused by intestinal helminths in a patient in need thereof with benzoyl peroxide, said method comprising topically administering to an intestinal helminths affected skin surface of said patient a therapeutically effective amount of a composition comprising from about 1% w/w to about 10% w/w or from about 3% w/w to about 7% w/w benzoyl peroxide in combination with at least one of a second active agent selected from Ivermectin, Mebendazole, Albendazole, pyrantel pamoate, Praziquantel, niclosamide, metrifonate, oxamniquine, bithionol, vermicides, vermifuges, anti-mite agent and any combination thereof. Each represents a separate embodiment of this invention. In another embodiment, the gastrointestinal disorder is enterobiasis.

In some other embodiments, there is provided a method of treating, preventing, alleviating intestinal helminths in a patient in need thereof with benzoyl peroxide, said method comprising topically administering to an intestinal helminths affected skin surface of said patient a therapeutically effective amount of a composition comprising from about 1% w/w to about 10% w/w or from about 3% w/w to about 7% w/w benzoyl peroxide in combination with at least one of a second active agent selected from Ivermectin, Mebendazole, Albendazole, pyrantel pamoate, Praziquantel, niclosamide, metrifonate, oxamniquine, bithionol, vermicides, vermifuges, anti-mite agent and any combination thereof.

### Regimen of Administration

In some embodiments of the method described hereinabove, the treatment comprises of at least two doses of administration, wherein the consecutive dose is administered within 7-14 days following the previous administration. In another embodiment, the consecutive dose is administered within 7, 8, 9, 10, 11, 12, 13 or 14 days following the previous administration.

In some embodiments of the method described hereinabove, the treatment comprises two doses of administration, wherein the second dose is administered within 7-14 days following the first administration. In another embodiment, the second dose is administered within 7, 8, 9, 10, 11, 12, 13 or 14 days following the first administration.

Another embodiment of the invention is a regimen of administration for treating, preventing or ameliorating of a gastrointestinal disorder caused by one or more intestinal helminths, wherein the regimen comprises at least two doses of administration of a topical composition comprising a peroxide and a carrier suitable for topical administration, wherein the consecutive dose is administered within 7-14 days following the previous administration, until remission.

In other embodiments, the regimen comprises two, three, four or more doses of administration.

In another embodiment, the invention provides a regimen of administration for treating, preventing or ameliorating of a gastrointestinal disorder caused by one or more intestinal helminths, wherein the regimen comprises two doses of administration of a topical composition comprising a peroxide and a carrier suitable for topical administration, wherein the consecutive dose is administered within 7-14 days following the previous administration, until remission.

### Definitions

Unless defined otherwise, all technical and scientific terms used herein have the same meaning as is commonly understood by one of skill in the art to which the invention pertains. In case of conflict, the specification, including definitions, takes precedence.

As used herein, the indefinite articles “a” and “an” mean “at least one” or “one or more” unless the context clearly dictates otherwise.

As used herein that “enterobiasis” and “a pinworm infection” can be used interchangeably. As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition. In another embodiment, “preventing” refers to preventing the disease and preventing others to be infected by an infected person.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein the term “treating gastrointestinal disorder caused by intestinal helminths”, refers to killing of intestinal helminths, inhibiting their growth, preventing spread thereof, preventing appearance of intestinal helminths thereof in a subject intestine.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

### Preparation and Composition of BPO Cream

TABLE 1
Ingredients used in the preparation of
BPO cream composition (100 gr cream):

| Ingredient | Weight (gr) | Function |
|---|---|---|
| Purified water | 46.8 | Solvent |
| BPO | 5.0 | API |
| Mineral oil | 15.0 | Solvent, Emollient |
| Stearic acid | 10.0 | Emulsifier |
| Cetyl alcohol | 7.0 | Stiffening agent |
| Sorbitol | 5.0 | Humectant |
| Polyethylene glycol 1000 | 5.0 | Emulsifier |
| Isopropyl myristate | 6.0 | Emollient |
| Propyl paraben | 0.18 | Preservative |
| Methyl paraben | 0.02 | Preservative |

### Preparation (100 gr Cream):

### Oil Phase:

Into a glass beaker 15.0 g of Mineral oil were added. The mineral oil was heated to 70° C.

7.0 g Cetyl alcohol, 5.0 gr of Polyehylene glycol 1000 and 10.0 gr of Stearic acid were added and mixed until complete dissolution.

### Water Phase:

Into a glass beaker 46.8 g Purified water were added. 6.0 g Isopropyl myristate, 0.18 g Propyl paraben, 0.02 g Methyl paraben and 5.0 g of Sorbitol were added. The water phase were heated to 70° C. and mixed until complete dissolution.

The oil phase was added slowly to the water phase under homogenization rate 17,000 rpm and mixing at 200 rpm. The homogenization and mixing were continued for another 10 min. The emulsion was cooled to 65° C. while mixing at 150 rpm. 5.0 g of BPO was added to the emulsion and the mixing was continued for another 30 min.

### Example 2

### 5% BPO Cream for Treating Pinworm Infection

A 4 years old girl diagnosed with pinworm infection was treated before bedtime with an encapsulated 5% BPO cream. The second dose was administered 1 week after the first dose. An immediate relief was reported after the administration of the first dose and no irritation was observed or reported. The second dose was given following complain of itching. No pinworms were observed during the following month after the second dose.

What is claimed is:

1. A method of treating, preventing and/or ameliorating a gastrointestinal disorder caused by one or more intestinal helminths, comprising administering topically to a subject in need thereof
a therapeutically effective amount of a topical composition, wherein the topical composition comprises a peroxide and a carrier suitable for topical administration.

2. The method according to claim 1, wherein the peroxide is benzoyl peroxide.

3. The method according to claim 1, wherein said gastrointestinal disorder is a pinworm infection (enterobiasis).

4. The method according to claim 2, wherein said gastrointestinal disorder is a pinworm infection (enterobiasis).

5. The method according to claim 1, wherein the peroxide is in an amount of about 1% w/w to about 10.0% w/w.

6. The method according to claim 1, wherein the peroxide is in an amount of 3%, 5% or 7% w/w.

7. The method according to claim 6, wherein the peroxide is benzoyl peroxide and is in an amount of 5% w/w.

8. The method according to claim 1, wherein said peroxide is encapsulated or non-encapsulated.

9. The method according to claim 1, wherein the peroxide is benzoyl peroxide, and the benzoyl peroxide is encapsulated.

10. The method according to claim 9, wherein the benzoyl peroxide is encapsulated by silica.

11. The method according to claim 1, wherein the treatment further comprises administering at least one additional active agent.

12. The method according to claim 11, wherein the additional active agent is selected from Ivermectin, Mebendazole, Albendazole, pyrantel pamoate, Praziquantel, niclosamide, metrifonate, oxamniquine, bithionol, vermicides, vermifuges, anti-mite agents, and any combination thereof.

13. The method according to claim 11, wherein the additional active agent is administered systematically.

14. The method according to claim 1, wherein the treatment comprises at least two doses of administration, wherein the consecutive dose is administered within 7-14 days following the previous administration.

15. The method according to claim 1, wherein the treatment comprises two doses of administration, wherein the second dose is administered within 7-14 days following the first administration.

16. A regimen of administration for treating, preventing or ameliorating a gastrointestinal disorder caused by one or more intestinal helminths, wherein the regimen comprises at least two doses of administration of a topical composition comprising a peroxide and a carrier suitable for topical administration, wherein the consecutive dose is administered within 7-14 days following the previous administration, until remission.

17. A regimen of administration for treating, preventing or ameliorating a gastrointestinal disorder caused by one or more intestinal helminths, wherein the regimen comprises two doses of administration of a topical composition comprising a peroxide and a carrier suitable for topical administration, wherein the consecutive dose is administered within 7-14 days following the previous administration, until remission.
